SEQUENCE LISTING (28 sequences)

SEQ ID NO: 1 - DNA, 56 bp, Artificial
  PRCR primer
ggatcctttattttcttattatttcttactctcactagtggtagtgaccttgaccg

SEQ ID NO: 2 - DNA, 53 bp, Artificial
  PCR primer
tgagtgtaattaggagcttgaacatcatcaaaagtggtacaacggtcaaggtc

SEQ ID NO: 3 - DNA, 58 bp, Artificial
  PCR primer
aattacactcaacatacttcatctatgcgtggggtttactatcctgatgaaatttttc

SEQ ID NO: 4 - DNA, 54 bp, Artificial
  PCR primer
aaaatggaagaaataaatcctgagttaaataaagagtgtctgaacgaaaaattt

SEQ ID NO: 5 - DNA, 57 bp, Artificial
  PCR primer
cttccattttattctaatgttactgggtttcatactattaatcatacgtttggcaac

SEQ ID NO: 6 - DNA, 54 bp, Artificial
  PCR primer
ggcagcaaaataaataccatccttaaaaggaatgacagggttgccaaacgtatg

SEQ ID NO: 7 - DNA, 53 bp, Artificial
  PCR primer
atttattttgctgccacagagaaatcaaatgttgtccgtggttgggtttttgg

SEQ ID NO: 8 - DNA, 57 bp, Artificial
  PCR primer
ggtaccaagcttattacacagactgtgacttgttgttcatggtagaaccaaaaaccc

SEQ ID NO: 9 - DNA, 57 bp, Artificial
  PCR primer
ggatccgtttgtggtccaaaattatctactgaccttattaagaaccagtgtgtcaat

SEQ ID NO: 10 - DNA, 58 bp, Artificial
  PCR primer
gaagaaggagttaacacaccagtaccagtgagaccattaaaattaaaattgacacact

SEQ ID NO: 11 - DNA, 57 bp, Artificial
  PCR primer
aactccttcttcaaagcgttttcaaccatttcaacaatttggccgtgatgtttctga

SEQ ID NO: 12 - DNA, 54 bp, Artificial
  PCR primer
ctaaaatttcagatgttttaggatcacgaacagaatcagtgaaatcagaaacat

SEQ ID NO: 13 - DNA, 53 bp, Artificial
  PCR primer
ctgaaattttagacatttcaccttgtgcttttgggggtgtaagtgtaattaca

SEQ ID NO: 14 - DNA, 58 bp, Artificial
  PCR primer
ggtaccaagcttattaaacagcaacttcagatgaagcatttgtaccaggtgtaattac

SEQ ID NO: 15 - DNA, 27 bp, Artificial
  PCR primer (SBC sense)
cgcggatccctcaagtatgatgaaaat

SEQ ID NO: 16 - DNA, 27 bp, Artificial
  PCR primer (SBC anti-sense)
cggggtaccttaaacagcaacttcaga

SEQ ID NO: 17 - DNA, 56 bp, Artificial
  PCR primer
ggatcccctcaaggtacaacattgccaaaaggcttctacgcagagggtagccgtgg

SEQ ID NO: 18 - DNA, 54 bp, Artificial
  PCR primer
accacgactacgtgatgaagaacgagaagaggcttgactgccgccacggctacc

SEQ ID NO: 19 - DNA, 53 bp, Artificial
  PCR primer
cacgtagtcgtggtaattcacgtaattcaactcctggcagcagtcgtggtaat

SEQ ID NO: 20 - DNA, 54 bp, Artificial
  PCR primer
gcgagggcagtttcaccaccaccgctagccatacgagcaggagaattaccacga

SEQ ID NO: 21 - DNA, 53 bp, Artificial
  PCR primer
gaaactgccctcgcacttttgctgcttgaccgtttgaaccagcttgagagcaa

SEQ ID NO: 22 - DNA, 54 bp, Artificial
  PCR primer
tagtgacagtttgaccttgttgttgttggcctttaccagaaactttgctctcaa

SEQ ID NO: 23 - DNA, 57 bp, Artificial
  PCR primer
caaactgtcactaagaaatctgctgctgaggcatctaaaaagcctcgtcaaaaacgt

SEQ ID NO: 24 - DNA, 59 bp, Artificial
  PCR primer
ggaccacgacgcccaaatgcttgagtgacgttgtactgttttgtggcagtacgtttttg

SEQ ID NO: 25 - DNA, 57 bp, Artificial
  PCR primer
gggcgtcgtggtccagaacaaacccaaggtaatttcggggaccaagaccttatccgt

SEQ ID NO: 26 - DNA, 59 bp, Artificial
  PCR primer
ggtaccaagcttattaaatttgcggccaatgtttgtaatcagtaccttgacggataagg

SEQ ID NO: 27 - DNA, 27 bp, Artificial
  PCR primer (N sense)
cgcggatcctctgataatggtccgcaa

SEQ ID NO: 28 - DNA, 30 bp, Artificial
  PCR primer (N anti-sense)
cggggtaccttaaatttgcggccaatgttt

(19) United States
(12) Patent Application Publication
(10) Pub. No.: US 2006/0140971 A1
(43) Pub. Date: Jun. 29, 2006

(54) Cell surface expression vector of sars virus antigen and microorganisms transformed thereby

(76) Inventors: Moon-Hee Sung, Daejeon (KR); Chul-Joong Kim, Daejeon (KR); Chang-Min Jung, Seoul (KR); Seung-Pyo Hong, Daejeon (KR); Jong Su Lee, Gyeonggi-do (KR); Jae Chul Choi, Daegu (KR); Kwang Kim, Daejeon (KR); Kuroda Shunichi, Osaka (JP); Ha Ryoung Poo, Daejeon (KR)

Correspondence Address: INTELLECTUAL PROPERTY / TECHNOLOGY LAW, PO BOX 14329, RESEARCH TRIANGLE PARK, NC, 27709, US

(21) Appl. No.: 10/559,631

(22) PCT Filed: Jun. 4, 2004
(86) PCT No.: PCT/KR04/01341

§ 371 Date: Dec. 3, 2005

(30) Foreign Application Priority Data
Jun. 4, 2003 (KR) 10-2003-0035993

(51) Int. Cl. A61K 39/02; C12N 1/21; C12N 15/74; C07K 14/165

(52) U.S. Cl. 424/190.1; 435/252.3; 530/350; 435/471

(57) ABSTRACT

The present invention relates to a surface expression vector of SARS coronavirus antigen containing a gene encoding an antigen of SARS inducing coronavirus and any one or two or more of genes pgsB, pgsC and pgsA encoding poly-gamma-glutamic acid synthase complex, a microorganism transformed by the surface expression vector, and a SARS vaccine comprising the microorganism. According to the present invention, it is possible to economically produce a vaccine for prevention and treatment of SARS using a recombinant strain expressing an SARS coronavirus antigen on their surface.

### TECHNICAL FIELD

The present invention relates to a vector expressing antigens of SARS on the surface of a microorganism, a microorganism transformed by the vector, and a vaccine for prevention of SARS comprising the transformed microorganism or an extracted and purified substance thereof. More particularly, it relates to a surface expression vector containing a gene encoding antigen proteins of SARS inducing coronavirus and any one or two or more of genes pgsB, pgsC and pgsA encoding poly-gamma-glutamic acid synthase complex which is a microorganism surface anchoring motif, a microorganism transformed by the vector, and a SARS vaccine comprising the transformed microorganism as an effective ingredient.

### BACKGROUND ART

Severe Acute Respiratory Syndrome (SARS) is a new type of an epidemic which has spread all over the world including Hong Kong, Singapore, Canada (Toronto) and so forth since it firstly broke out in November 2002 centering around Guangdong province in China. It shows respiratory symptoms such as fever of 38° C. or higher and coughing, dyspnoea, atypical pneumonia. The agent of SARS is known as a mutant pathogenic coronavirus.

Generally, the members of coronavirus family are very large RNA viruses having (+)RNA. The genome is composed of about 29,000 to 31,000 bases and observed as a crown shape under a microscope. It contributes to upper respiratory diseases in human, respiratory, liver, nerves and intestines related diseases in animals. Three groups of coronavirus exist in nature. Among them, group I and group II infect mammals and group III infects birds.

The known coronavirus in nature sometimes induce lung related diseases in persons with weakened immune system or cause severe diseases in animals such as dogs, cats, pigs, mice, birds and the like. They show a very high mutation rate and a high recombination rate of about 25%. It is presumed that such properties cause mutation of original coronavirus, to produce a novel mutant coronavirus (SARS coronavirus), which is propagated from animals to human.

According to World Health Organization (WHO), 7,447 suspected SARS patients in 31 countries have identified since November, 2002 and 551 of them died. The SARS infection danger zone of 2003 include Beijing, Guangdong, Hong Kong, inner Mongolia, Shanxi and Tianjin in China, Singapore, Toronto in Canada, Taiwan, Ulanbaator in Mongol, Philippines and the like. However, this has a risk to be spread all over the world.

Since the outbreak on 2002, as to SARS coronavirus, a Germany institute for tropical medicine firstly performed decoding of the nucleotide sequence of SARS virus. The research team decoded the nucleotide sequence of a specific genetic part where the amplification by PCR (Polymerase Chain Reaction) can be done. The decoded result was given to Artus GmbH which is a bioengineering company in Germany and used to develop a kit to detect infection of SARS. This kit can determine the infection of SARS virus by amplification of virus gene from a suspected SARS patient.

Thereafter, the whole genome of SARS virus was decoded and up to now, the sequences of more than 12 isolate strains are completely analyzed. The whole sequence of Urbani strain, which is the firstly isolated strain [dubbing the name of the WHO mission doctor who died of SARS, SARS-Cov strain (Rota, Pa., Science 108:5952, 2003; GenBank Accession AY278741)] was decoded by a CDC research team of USA. The Canada British Columbia Cancer search center team analyzed the whole sequence of SARS Tor2 virus strain isolated from a patient in Toronto, Canada, on Apr. 12, 2003 (Marra, M. A., Science 108:5953, 2003; GenBank Accession 274119).

Though the two research teams analyzed coronavirus isolated from patients infected with SARS in each different place, the two viruses showed difference in only 15 bases. This suggests that SARS has been induced from the same virus. Also, according to the result of a genomic analysis of SARS coronavirus, it is known that it has the same components forming proteins as those of the existing coronavirus but shows little homology in genome and amino acids by genome. Rat hepatitis virus and turkey bronchitis virus show similarity to SARS coronavirus. However, the correlation of SARS coronavirus and other coronavirus is presented by molecular taxonomic analysis and it is concluded that SARS coronavirus is different from the existing groups.

At present, the detection of SARS coronavirus begins with PCR and the positive result of the antibody test is determined by ELISA or IFA. The virus isolation is performed by subjecting a subject identified by PCR to a cell culture test and determining the infection of SARS coronavirus.

There is no fundamental method for treating SARS but supplementary supporting therapy. The research on SARS coronavirus, which is an agent of the new epidemic, is in the beginning step and no vaccine for prevention was developed. Diversified researches are being conducted to develop a vaccine for prevention all over the world.

The technology to attach and express a desired protein onto the cell surface of a microorganism is called as cell surface display technology. The cell surface display technology uses surface proteins of microorganisms such as bacteria or yeast as a surface anchoring motif to express a foreign protein on the surface and has an application scope including production of recombinant live vaccine, construction of peptide/antibody library and screening, whole cell absorbent, whole cell biotransformation catalyst and the like. The application scope of this technology is determined by a protein to be expressed on the cell surface. Therefore, the cell surface display technology has tremendous potential of industrial applicability.

For successive cell surface display technology, the surface anchoring motif is the most important. It is the core of this technology to select and develop a motif expressing a foreign protein on the cell surface effectively.

Therefore, in order to select a surface anchoring motif, the following properties should be considered. (1) It should have a secretion signal to help a foreign protein to pass through the cellular inner membrane so that the foreign protein can be transferred to the cell surface. (2) It should have a target signal to help a foreign protein to be stably fixed on the surface of the cellular outer membrane. (3) It can be expressed in a large quantity on the cell surface but does not affect growth of the cell. (4) It has nothing to do with protein size and can express a foreign protein without change in the three-dimensional structure of the protein. However, a surface anchoring motif satisfying the foregoing requirements has not yet been developed.

The surface anchoring motives which have been known and used so far are largely classified into four types of cell outer membrane proteins, lipoproteins, secretory proteins, surface organ proteins such as flagella protein. In case of gram negative bacteria, proteins existing on the cellular outer membrane such as LamB, PhoE (Charbit et al., J. Immunol., 139:1658, 1987; Agterberg et al., Vaccine, 8:85, 1990), OmpA and the like have been used. Also, lipoproteins such as TraT (Felici et al., J. Mol. Biol., 222:301, 1991), PAL (peptidoglycan associated lipoprotein) (Fuchs et al., Bio/Technology, 9:1369, 1991) and Lpp(Francisco et al., Proc. Natl. Acad. Sci. USA, 489:2713, 1992) have been used. Fimbriae proteins such as FimA or FimH adhesion of tppe 1 fimbriae (Hedegaard et al., Gene, 85:115, 1989), pili proteins such as PapA pilu subunit have been used as a surface anchoring motif to attempt expression of a foreign protein. In addition, it has been reported that ice nucleation protein (Jung et al., Nat. Biotechnol., 16:576, 1998; Jung et al., Enzyme Microb. Technol., 22:348, 1998; Lee et al., Nat. Biotechnol., 18:645, 2000), pullulanase of Klebsiela oxytoca (Kornacker et al., Mol. Microl., 4:1101, 1990), IgA protease of Neiseria (Klauser et al., EMBO J., 9:1991, 1990), AIDA-1, which is adhesion of E. coli, VirG protein of shigella, a fusion protein of Lpp and OmpA may be used as a surface anchoring motif. Upon use of gram positive bacteria, there have been reported that malaria antigen was effectively expressed using Staphylococcus aureus derived protein A and FnBPB protein as a surface anchoring motif, a surface coat protein of lactic acid bacteria used in surface expression, and surface proteins of gram positive bacteria such as Streptococcus pyogenes derived M6 protein (Medaglini, D et al., Proc. Natl. Acad. Sci. USA., 92:6868, 1995), Bacillus anthracis derived S-layer protein EA1, Bacillus subtilis CotB and the like were used as a motif.

The present inventors have developed a novel vector for effectively expressing a foreign protein on the cell surface of a microorganism by using poly-gamma glutamic acid synthesizing complex gene (pgsBCA) derived from Bacillus genus strain as a novel surface anchoring motif and a method for mass-expressing a foreign protein on the surface of a microorganism transformed by the vector (Korean Patent Application No. 10-2001-48373).

Researches have been conducted to stably express a pathogenic antigen or an antigen determining group in bacteria suitable for mass-production by genetic engineering method using the above-listed surface anchoring motives. Particularly, it has been reported that an exogenous immunogen expressed on the surface non-pathogenic bacteria, when being orally administered in the live state, can induce more sustained and stronger immune response, as compared to vaccines using attenuated pathogenic bacteria or viruses. Such induction of immune response is attributable to the adjuvant action of the surface structures of bacteria to increase antigenicity of the foreign protein expressed on the surface and immune response to the live bacteria in the living body. The development of a recombinant live vaccine of non-pathogenic bacteria using this surface expression system has attracted public attention.

Therefore, the present inventors have succeeded in mass-expressing antigens of SARS coronavirus chosen by gene and protein analyses on the surface of a non-pathogenic microorganism, of which food safety is secured, such as lactic acid bacteria by using poly-gamma-glutamic acid synthesizing complex gene (pgsBCA) derived from Bacillus genus strain as a surface anchoring motif and developed an economic and stable vaccine to induce production of antibody to SARS coronavirus in blood and mucosal immunization through oral administration of the microorganism.

### DISCLOSURE OF INVENTION

Therefore, it is an object of the present invention to provide a vector capable of expressing a SARS coronavirus antigen by employing a surface expression system of a microorganism and a microorganism transformed by the vector.

It is another object of the present invention to provide a transformed microorganism having an antigen of SARS coronavirus expressed on the surface, a vaccine for prevention of SARS comprising a SARS coronavirus antigen extracted from the microorganism or a SARS coronavirus antigen purified from the microorganism as an effective ingredient.

In order to accomplish the above objects, according to the present invention, there is provided a surface expression vector comprising any one or two or more of pgsB, pgsC and pgsA genes encoding poly-gamma-glutamic acid synthase complex and a gene encoding a spike antigen protein or a nucleocapsid antigen protein of SARS coronavirus.

According to the present invention, as the surface antigen protein gene, any gene encoding a spike antigen protein of SARS coronavirus can be used. It is possible to use a spike antigen protein gene of SARS coronavirus alone or as a complex of two or more. Also, the gene encoding the poly-gamma-glutamic acid synthase complex preferably includes pgsA. The spike antigen protein may be SARS SA, SARS SB, SARS SC, SARS SD or SARS SBC and the nucleocapsid antigen protein may be SARS NA, SARS NB or SARS N.

Also, the present invention provides a microorganism transformed by the expression vector and a method for producing a spike antigen protein or a nucleocapsid antigen protein of SARS coronavirus comprising culturing the microorganism.

The microorganism applicable to the present invention may be any microorganism which does not show toxicity upon application to a living body, or any attenuated microorganism. For example, it can be properly selected from gram negative bacteria, such as E. coli, Salmonella typhi, Salmonella typhimurium, Vibrio cholerae, Mycobacterium bovis, Shigella and the like or gram positive bacteria such as Bacillus, Lactobacillus, Lactococcus, Staphylococcus, Listeria monocytogenes, Streptococcus and the like. Selection of an edible microorganism such as lactic acid bacteria is particularly preferred.

Further, the present invention provides a vaccine for prevention of SARS comprising a microorganism having the antigen protein expressed on the surface, a crude form extracted from cell membrane components of the microorganism which has been broken, or an antigen protein purified from the microorganism as an effective ingredient.

The vaccine according to the present invention can be used as a medicine for prevention of SARS (Severe Acute Respiratory Syndrome) induced by SARS coronavirus.

The vaccine according to the present invention can be taken by oral administration or in food, subcutaneously or intra-peritoneally injected, or administered by the intranasal route.

Up to date; the infection of SARS coronavirus is known to be induced by infection of a respiratory organ by infectious droplets and presumed to occur at the mucosal surface of the respiratory organ. Thus, the protection of infection by mucosal immunity is very important. Since the microorganism expressing an antigen of SARS coronavirus on the surface has an advantage that can more effectively induce antibody formation on a mucous membrane (mucosal response), the vaccine for oral administration or the vaccine for intranasal administration using the transformed microorganism is expected to be more effective than a parenteral vaccine in the protection against SARS coronavirus.

### BRIEF DESCRIPTION OF DRAWINGS

Further objects and advantages of the invention can be more fully understood from the following detailed description taken in conjunction with the accompanying drawings.

FIG. 1 shows the relations between four antigenic sites (A, B, C, D) of swine transmissible gastro enteritis virus and the spike protein of SARS coronavirus by hydrophilicity plot according to the Kyte-Doolittle method, antigenic index according to the Jameson-wolf method and surface probability plot according to the Emini method.

FIG. 2 shows the relation between the nucleocapsid protein of swine transmissible gastro enteritis virus and the nucleocapsid protein of SARS coronavirus by hydrophilicity plot according to the Kyte-Doolittle method, antigenic index according to the Jameson-wolf method and surface probability plot according to the Emini method.

FIG. 3A is a genetic map of the vector pHCE2LB:pgsA-SARS SA for surface expression comprising the gram negative and gram positive microorganisms as a host according to the present invention, FIG. 3B is a genetic map of pHCE2LB:pgsA-SARS SC according to the present invention and FIG. 3C is a genetic map of pHCE2LB:pgsA-SARS SBC according to the present invention.

FIG. 4A is a genetic map of the vector pHCE2LB:pgsA-SARS NB according to the present invention and FIG. 4B is a genetic map of pHCE2LB:pgsA-SARS N according to the present invention.

FIGS. 5A, 5B and SC are to identify expression of the SARS SA, SARS SC and SARS SBC antigens fused with the cellular outer membrane protein pgsA in Lactobacillus by showing the specific bonding between a specific antibody to pgsA and the fusion proteins by Western immunoblotting.

FIGS. 6A and 6B are to identify surface expression of the SARS SA and SARS SBC antigens fused with the cellular outer membrane protein pgsA in Lactobacillus by performing Western immunoblotting using proteins fragmented from lactic acid bacteria cells as a specific antibody to pgsA and FIG. 6C is to identify surface expression of the SARS SBC antigen in Lactobacillus by FACScan assay.

FIGS. 7A and 7B are to identify surface expression of the SARS NB and SARS N antigens fused with the cellular outer membrane protein pgsA in Lactobacillus by performing Western immunoblotting using proteins fragmented from lactic acid bacteria cells as a specific antibody to pgsA.

FIG. 8 shows the results of measurement of IgG antibody value to the SARS SA and SARS SC antigens in serum of mouse which has been orally and intranasally administered with the Lactobacillus casei strains, which are each transformed with the vectors pHCE2LB:pgsA-SARS SA, pHCE2LB:pgsA-SARS SC and pHCE1LB:pgsA-SARS NB for surface expression according to the present invention and have the surface expression of the antigen group identified by ELISA (Enzyme-linked Immunosorbent Assay).

FIG. 9 shows the results of measurement of IgA antibody value to the SARS SA and SARS SC antigens in the intestine washing liquid and bronchus-alveolar washing liquid of mouse which has been orally and intranasally administered with the Lactobacillus casei strains, which are each transformed with the vectors pHCE2LB:pgsA-SARS SA, pHCE2LB:pgsA-SARS SC and pHCE1LB:pgsA-SARS NB for surface expression according to the present invention and have the surface expression of the antigen group identified, by ELISA.

FIG. 10 shows the results of measurement of IgG antibody value to the SARS NB antigen group in serum of mouse which has been orally and intranasally administered with the Lactobacillus casei strains, which are each transformed with the vectors pHCE2LB:pgsA-SARS SA, pHCE2LB:pgsA-SARS SC and pHCE1LB:pgsA-SARS NB for surface expression according to the present invention and have the surface expression of the antigen group identified, by ELISA.

FIG. 11 shows the results of measurement of IgA antibody value to the SARS NB antigen group in the intestine washing liquid and bronchus-alveolar washing liquid of mouse which has been orally and intranasally administered with the Lactobacillus casei strains, which are each transformed with the vectors pHCE2LB:pgsA-SARS SA, pHCE2LB:pgsA-SARS SC and pHCE1LB:pgsA-SARS NB for surface expression according to the present invention and have the surface expression of the antigen group identified, by ELISA.

### BEST MODE FOR CARRYING OUT THE INVENTION

The Now, the present invention will be explained in further detail by the following examples. It is apparent to those possessing ordinary knowledge in the art that the examples are only for concrete explanation of the present invention and the scope of the present invention is not limited thereto.

Particularly, though genes of an antigenic site in the spike protein of SARS coronavirus and genes of an antigenic site in the nucleocapsid protein of SARS coronavirus are applied in the following examples, any antigen protein gene may be used alone or as a complex of two or more.

Also, in the following examples, the gene pgsBCA of the cellular outer membrane protein which is involved in synthesis of poly-gamma-glutamic acid is obtained from Bacillus subtilis var. chungkookjang (KCTC 0697BP) and used. However, according to the present invention, the gene includes vectors prepared using pgsBCA obtained from all Bacillus genus strains producing poly-gamma-glutamic acid or microorganisms transformed with those vectors. For example, preparation of a vector for a vaccine using the pgsBCA gene derived from other strains having homology of 80% or more with the sequence of the pgsBCA gene existing in Bacillus subtilis var. chungkookjang and use of the vector are included in the scope of the present invention.

Further, in the following examples, only pgsA of the gene pgsBCA is used to construct a vector for surface expression. However, as can be inferred from indirect examples, use of the whole or a part of the gene pgsBCA to construct a vector for a vaccine is included in the scope of the present invention.

In the following examples, Salmonella typhi, which is a gram negative bacterium and Lactobacillus, which is a gram positive bacterium are used as a host for the vector. However, it becomes apparent to those skilled in the art that any kind of gram negative bacteria or gram positive bacteria which have been transformed by the method according to the present invention can provide the same results.

In addition, in the following examples, only cases applying a microorganism itself transformed by the vector for a vaccine according to the present invention as a live vaccine to a living body are presented. However, according to the knowledge of the vaccine-related technical field, it is natural to have identical or similar results even when expression proteins (antigen proteins of SARS coronavirus) crudely extracted from the microorganism or purified expression proteins are applied to a living body.

### EXAMPLE 1

### Synthesis of Antigenic Site Gene in Spike Protein of SARS Coronavirus

The spike protein of SARS coronavirus is a glycoprotein composed of 1256 amino acids. In case of other coronavirus which have been much examined, the spike protein is mostly inserted into an envelope protein. covering the surface of a virus particle to have a structure exposed to the outside. The exposed site and the antigenic site have been intensively studied as a target antigen of a vaccine to induce virus infection and to prevent the infection.

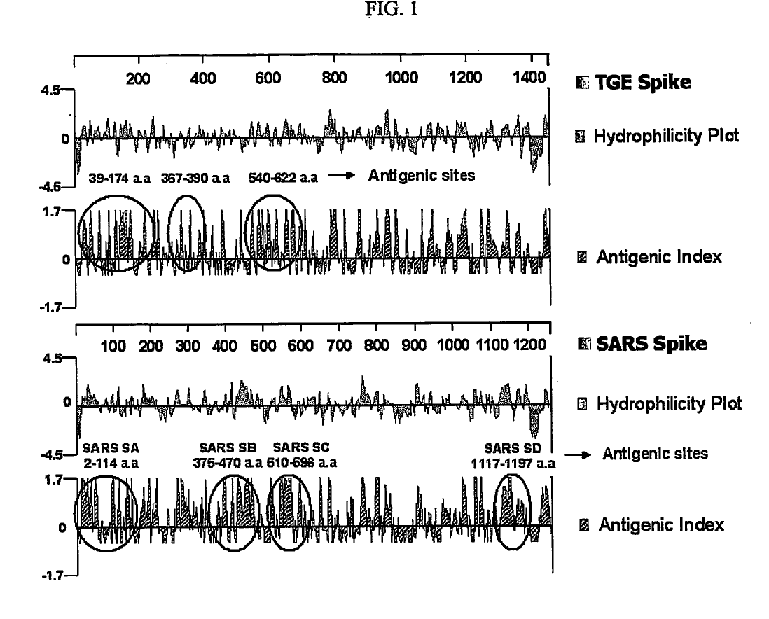

Therefore, in order to select a site capable of showing antigenicity from the 1256 amino acids of the spike protein of SARS coronavirus, the antigenic site was chosen by comparative analysis of proteins and structural comparative analysis with the spike protein of other swine transmissible gastroenteritis (TGE) coronavirus which has been studied for antigenicity and synthesized. Concretely, the antigenic site of the spike protein of swine transmissible gastroenteritis virus is well known as four sites (A, B, C, D) (Enjuanes, L., Virology, 183:225, 1991). The relation between these sites and the spike protein of SARS coronavirus was analyzed by hydrophilicity plot according to the Kyte-Doolittle method, antigenic index according to the Jameson-wolf method and surface probability plot according to the Emini method and SARS SA, SARS SB, SARS SC and SARS SD were selected from the sequence of the spike protein of SARS coronavirus Tor2 isolate (FIG. 1).

Firstly, based on the sequence of the spike protein of SARS coronavirus Tor2 isolate (21492-25259 bases, 1255 amino acids), of which the whole sequence had been identified, the 2 to 114 amino acid site which was expected to be an antigenic site was selected and denominated SARS SA, the 375 to 470 amino acid site was selected and denominated SARS SB, the 510 to 596 amino acid site was selected and denominated SARS SC, and the 1117 to 1197 amino acid site was selected and denominated SARS SD. Among these antigenic sites, genes of the SARS SA and SARS SC sites were synthesized.

In order to synthesize a gene corresponding to the 113 length amino acids denominated SARS SA, PCR was performed using primers of SEQ ID NOs: 1 to 8 to obtain the amplified SARS SA gene of 339 bp.

SEQ ID NO: 1: 5′-ggatcctttattttcttattatttcttactctcactagtggtagtgaccttgaccg-3′
SEQ ID NO: 2: 5′-tgagtgtaattaggagcttgaacatcatcaaaagtggtacaacggtcaaggtc-3′
SEQ ID NO: 3: 5′-aattacactcaacatacttcatctatgcgtggggtttactatcctgatgaaatttttc-3′
SEQ ID NO: 4: 5′-aaaatggaagaaataaatcctgagttaaataaagagtgtctgaacgaaaaattt-3′
SEQ ID NO: 5: 5′-cttccattttattctaatgttactgggtttcatactattaatcatacgtttggcaac-3′
SEQ ID NO: 6: 5′-ggcagcaaaataaataccatccttaaaaggaatgacagggttgccaaacgtatg-5′
SEQ ID NO: 7: 5′-atttattttgctgccacagagaaatcaaatgttgtccgtggttgggtttttgg-3′
SEQ ID NO: 8: 5′-ggtaccaagcttattacacagactgtgacttgttgttcatggtagaaccaaaaaccc-3′

In order to synthesize a gene corresponding to the 87 length amino acids denominated SARS SC, PCR was performed using primers of SEQ ID NOs: 9 to 14 to obtain the amplified SARS SC gene of 261 bp.

SEQ ID NO: 9: 5′-ggatccgtttgtggtccaaaattatctactgaccttattaagaaccagtgtgtcaat-3′
SEQ ID NO: 10: 5′-gaagaaggagttaacacaccagtaccagtgagaccattaaaattaaaattgacacact-3′
SEQ ID NO: 11: 5′-aactccttcttcaaagcgttttcaaccatttcaacaatttggccgtgatgtttctga-3′
SEQ ID NO: 12: 5′-ctaaaatttcagatgttttaggatcacgaacagaatcagtgaaatcagaaacat-3′
SEQ ID NO: 13: 5′-ctgaaattttagacatttcaccttgtgcttttgggggtgtaagtgtaattaca-3′
SEQ ID NO: 14: 5′-ggtaccaagcttattaaacagcaacttcagatgaagcatttgtaccaggtgtaattac-3′

In addition, the genes of the antigenic sites were obtained by synthesis, a gene encoding the site of 264 to 596 amino acids was amplified by PCR using the SARS spike cDNA clone (SARS coronavirus TOR2) from Canada's Michael Smith Genome Science Center as a template and primers of SEQ ID NOs: 15 and 16 to obtain a gene of 996 bp, which was denominated SARS SBC [this gene contains a critical site to produce a neutralizing antiby (PNAS, 101:2536, 2004)].

SEQ ID NO: 15 (SBC sense): 5′-cgcggatccctcaagtatgatgaaaat-3′
SEQ ID NO: 16 (SBC anti-sense): 5′-cggggtaccttaaacagcaacttcaga-3′

### EXAMPLE 2

### Synthesis of Antigenic Site Gene in Nucleocapsid Protein of SARS Coronavirus

The nucleocapsid protein of SARS coronavirus is a protein composed of 422 amino acids. It has been reported that most of the nucleocapsid proteins of other coronavirus on which much research has been conducted serve as an antigen. Such antigenic site has been intensively studied to use a target antigen of a vaccine to prevent the infection of coronavirus.

Therefore, sites capable of showing antigenicity in the amino acids of the nucleocapsid protein of SARS coronavirus was chosen by comparative analysis of proteins with the nucleocapsid protein of swine transmissible gastroenteritis (TGE) coronavirus and synthesized.

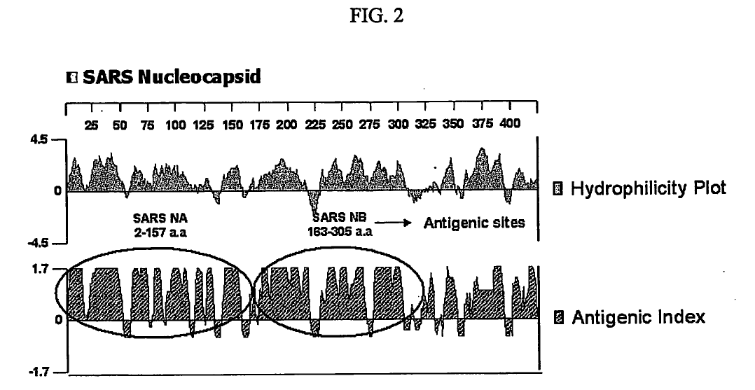

Concretely, the relation between the nucleocapsid protein of swine transmissible gastroenteritis virus and the nucleocapsid protein of SARS coronavirus was analyzed by hydrophilicity plot according to the Kyte-Doolittle method, antigenic index according to the Jameson-wolf method and surface probability plot according to the Emini method and SARS NA and SARS NB were selected from the sequence of the nucleocapsid protein of SARS coronavirus Tor2 isolate (FIG. 2).

Firstly, based on the sequence of the nucleocapsid protein of SARS coronavirus Tor2 isolate (28120-29388 bases, 422 amino acids), of which the whole sequence had been identified, the 2 to 157 amino acid site which was expected to be an antigenic site was selected and denominated SARS NA and the 163 to 305 amino acid site was selected and denominated SARS NB. In the present invention, the gene of the SARS NB site was synthesized.

In order to synthesize a gene corresponding to the 143 length amino acids denominated SARS NB, PCR was performed using primers of SEQ ID NOs: 17 to 26 to obtain the amplified SARS NB gene of 429 bp.

SEQ ID NO: 17: 5′-ggatcccctcaaggtacaacattgccaaaaggcttctacgcagagggtagccgtgg-3′
SEQ ID NO: 18: 5′-accacgactacgtgatgaagaacgagaagaggcttgactgccgccacggctacc-3′
SEQ ID NO: 19: 5′-cacgtagtcgtggtaattcacgtaattcaactcctggcagcagtcgtggtaat-3′
SEQ ID NO: 20: 5′-gcgagggcagtttcaccaccaccgctagccatacgagcaggagaattaccacga-3′
SEQ ID NO: 21: 5′-gaaactgccctcgcacttttgctgcttgaccgtttgaaccagcttgagagcaa-3′
SEQ ID NO: 22: 5′-tagtgacagtttgaccttgttgttgttggcctttaccagaaactttgctctcaa-3′
SEQ ID NO: 23: 5′-caaactgtcactaagaaatctgctgctgaggcatctaaaaagcctcgtcaaaaacgt-3′
SEQ ID NO: 24: 5′-ggaccacgacgcccaaatgcttgagtgacgttgtactgttttgtggcagtacgtttttg-3′
SEQ ID NO: 25: 5′-gggcgtcgtggtccagaacaaacccaaggtaatttcggggaccaagaccttatccgt-3′
SEQ ID NO: 26: 5′-ggtaccaagcttattaaatttgcggccaatgtttgtaatcagtaccttgacggataagg-3′

In addition, the genes of the antigenic sites were obtained by synthesis, a gene encoding the site of 2 to 305 amino acids was amplified by PCR using the SARS nucleocapsid cDNA clone (SARS coronavirus TOR2) from Canada's Michael Smith Genome Science Center as a template and primers of SEQ ID NOs: 27 and 28 to obtain a gene of 912 bp, which was denominated SARS N.

SEQ ID NO: 27 (N sense): 5′-cgcggatcctctgataatggtccgcaa-3′
SEQ ID NO: 28 (N anti-sense): 5′-cggggtaccttaaatttgcggccaatgttt-3′

### EXAMPLE 3

### Construction of pHCE2LB:pgsA-SARS SA and pHCE2LB:pgsA-SARS SC Vectors for Surface Expression

The surface expression vectors pHCE2LB:pgsA-SARS SA and pHCE2LB:pgsA-SARS SC capable of surface expressing the antigenic sites SARS SA and SC in the spike protein of SARS coronavirus were constructed using pgsA of the gene (pgsBCA) of the cellular outer membrane protein derived from Bacillus genus strain and participating in the synthesis of poly-gamma-glutamic acid and a gram negative microorganism and a gram positive microorganism as hosts.

Firstly, in order to introduce the antigenic sites SARS SA and SARS SC in the spike protein of SARS coronavirus to a vector for surface expression having the L1 antigen of human papilloma virus expressed with gram negative and gram positive microorganisms as hosts (a vector containing HCE promoter, which is a constantly high expression promoter, pgsA of the gene (pgsBCA) of the cellular outer membrane protein participating in the synthesis of poly-gamma-glutamic acid and HPV L1 in pAT which is a vector for general use for gram negative and gram positive bactera), pHCE2LB:pgsA-HPVL1 (KCTC 10349BP) was digested with BamHI and KpnI. The HPVL1 gene was removed to prepare a vector pHCE2LB:pgsA for surface expression.

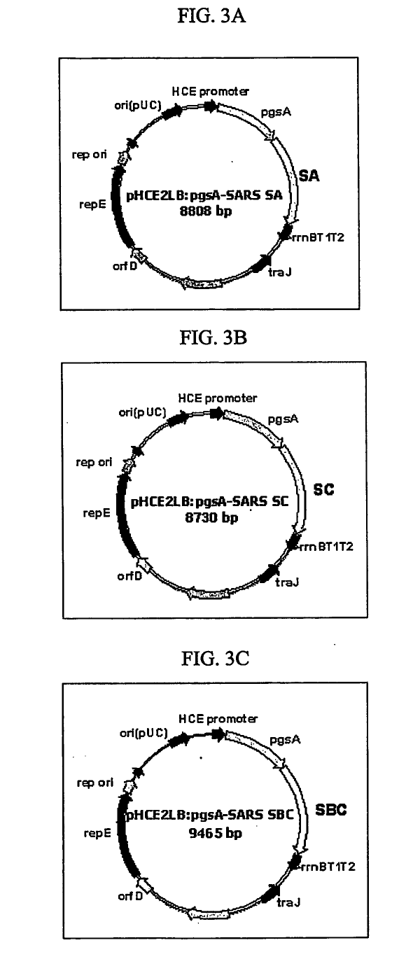

The SARS SA and SARS SC antigen genes synthesized in Example 1 were each digested with restriction enzymes BamHI and KpnI and joined to the C-terminal region of the gene pgsA of the cellular outer membrane protein participating in the synthesis of poly-gamma-glutamic acid of the previously prepared surface expression vector pHCE2LB:pgsA in accordance with the translation codon to prepare vectors pHCE2LB:pgsA-SARS SA and pHCE2LB:pgsA-SARS SC (FIGS. 3A and 3B). The gram positive bacterium Lactobacillus was transformed with the prepared surface expression vectors pHCE2LB:pgsA-SARS SA and pHCE2LB:pgsA-SARS SC, and the presence of pHCE2LB:pgsA-SARS SA and pHCE2LB:pgsA-SARS SC plasmids in Lactobacillus was examined.

### EXAMPLE 4

### Construction of pHCE2LB:pgsA:SARS SBC Vector for Surface Expression

The pHCE2LB:pgsA-SARS SBC vector capable of surface expressing the antigenic site SARS SBC in the spike protein of SARS coronavirus was constructed using pgsA of the gene (pgsBCA) of the cellular outer membrane protein derived from Bacillus genus strain and participating in the synthesis of poly-gamma-glutamic acid.

Firstly, by the method described in the Example 3, the surface expression vector pHCE2LB:pgsA was prepared. The gene encoding the 264-596 amino acid site was amplified by PCR using the SARS spike cDNA clone of SARS coronavirus, described in the Example 1, as a template to obtain SARS SBC gene of 996 bp. The SARS SBC gene was then inserted into the surface expression vector pHCE2LB:pgsA to prepare pHCE2LB:pgsA-SARS SBC (FIG. 3C). The gram positive bacterium Lactobacillus was transformed with the prepared surface expression vector pHCE2LB:pgsA-SARS SBC and the presence of pHCE2LB:pgsA-SARS SBC plasmid in Lactobacillus was examined.

### EXAMPLE 5

### Construction of pHCE2LB:pgsA:SARS NB Vector for Surface Expression

The pHCE2LB:pgsA-SARS NB vector capable of surface expressing the antigenic site SARS NB in the nucleocapsid protein of SARS coronavirus was constructed using pgsA of the gene (pgsBCA) of the cellular outer membrane protein derived from Bacillus genus strain and participating in the synthesis of poly-gamma-glutamic acid.

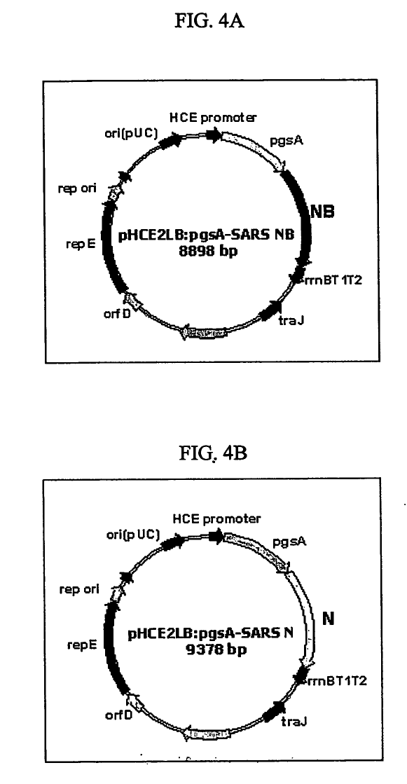

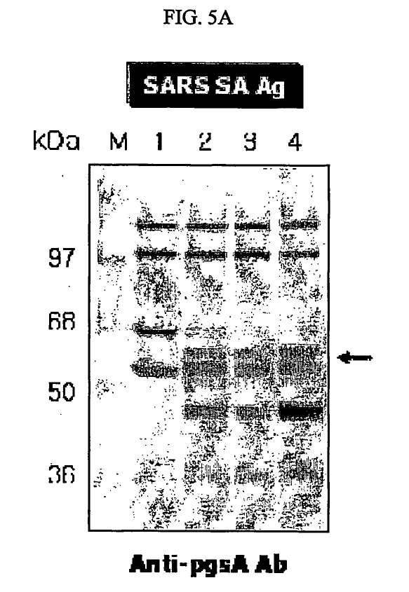

Firstly, by the method described in the Example 3, the surface expression vector pHCE2LB:pgsA was prepared. The SARS NB antigen gene synthesized in the Example 2 was digested with restriction enzymes BamHI and KpnI and joined to the C-terminal of the gene pgsA of the cellular outer membrane protein participating in the synthesis of poly-gamma-glutamic acid of the previously prepared surface expression vector pHCE2LB:pgsA in accordance with the translation codon to prepare a vector pHCE2LB:pgsA-SARS NB (FIG. 4A). The gram positive bacterium Lactobacillus was transformed with the prepared surface expression vector pHCE2LB:pgsA-SARS NB and the presence of pHCE2LB:pgsA-SARS NB plasmid in Lactobacillus was examined.

### EXAMPLE 6

### Construction of pHCE2LB:pgsA-SARS N Vector for Surface Expression

The pHCE2LB:pgsA-SARS N vector capable of surface expressing the antigenic site SARS N in the nucleocapsid protein of SARS coronavirus was constructed using pgsA of the gene (pgsBCA) of the cellular outer membrane protein derived from Bacilius genus strain and participating in the synthesis of poly-gamma-glutamic acid.

Firstly, by the method described in the Example 3, the surface expression vector pHCE2LB:pgsA was prepared. The gene encoding the 2305 amino acid site was amplified by PCR using the SARS nucleocapsid cDNA clone of SARS coronavirus, described in the Example 2, as a template to obtain SARS N gene of 912 bp. The SARS N gene was then inserted into the surface expression vector pHCE2LB:pgsA to prepare pHCE2LB:pgsA-SARS N (FIG. 4B). The gram positive bacterium Lactobacillus was transformed with the prepared surface expression vector pHCE2LB:pgsA-SARS N and the presence of pHCE2LB:pgsA-SARS N plasmid in Lactobacillus was examined.

### EXAMPLE 7

### Confirmation of Surface Expression of SARS Virus Spike Antigen Protein on Lactic Acid Bacteria

Lactobacillus was transformed with the surface expression vectors pHCE2LB:pgsA-SARS SA, pHCE2LB:pgsA-SARS SC and pHCE2LB:pgsA-SARS SBC and examined for expression of respective antigen proteins.

The expression of the antigenic sites in the spike antigen of SARS virus fused with the C-terminal of the gene pgsA synthesizing poly-gamma-glutamic acid was induced by transforming Lactobacillus casei with pHCE2LB:pgsA-SARS SA, pHCE2LB:pgsA-SARS SC and pHCE2LB:pgsA-SARS SBC, subjecting the transformed strain in MRS medium (Lactobacillus MRS, Becton Dickinson and Company Sparks, USA), to a stationary culture and multiplication at 37° C.

The expression of each spike antigen was identified by performing Western immunoblotting using SDS-polyacrylamide gel electrophoresis and a specific antibody to pgsA. The whole cells of Lactobacillus casei whose expression is induced concretely were denatured with proteins obtained at the same cell concentration to prepare samples. They were analyzed by SDS-polyacrylamide gel electrophoresis and the fractionated proteins were transferred to PVDF membrane (polyvinylidene-difluoride membranes, Bio-Rad). The PVDF membrane with the proteins transferred thereon in a blocking buffer solution (50 mM Tris HCl, 5% skim milk, pH 8.0) was blocked by shaking for 1 hour and reacted with rabbit-derived polyclone primary antibody to pgsA, which have been diluted 1000 times with the blocking buffer solution, for 12 hours.

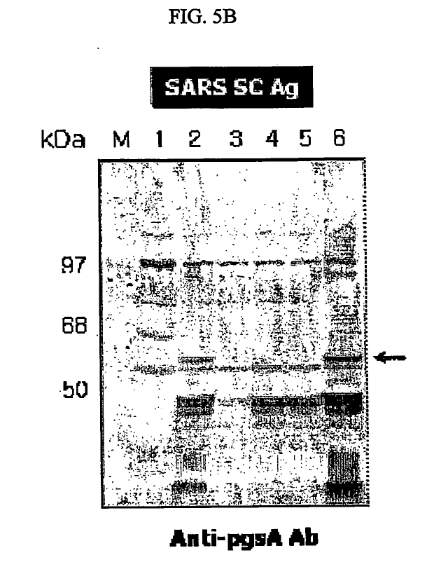

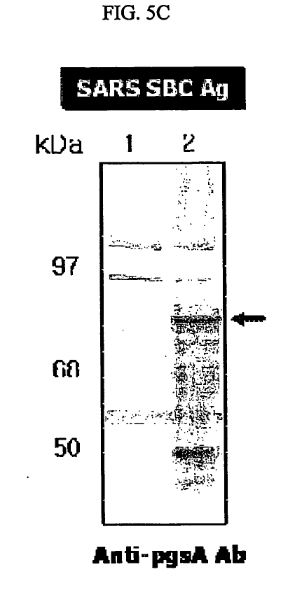

After completion of the reaction, the membrane was washed with buffer solution and reacted with biotin-binding secondary antibody to rabbit, which have been diluted 1000 times with the blocking buffer solution, for 4 hours. After completion of the reaction, the membrane was washed with buffer solution and reacted with a avidin-biotin reagent for 1 hour, followed by washing. The washed membrane was treated with H₂O₂ and DAB solution as a substrate and a color developing agent to confirm that the specific bonding between the specific antibody to pgsA and the fusion protein (FIG. 5). In FIG. 5A, lane 1 is non-transformed Lactobacillus casei, and lane 2, 3 and 4 are Lactobacillus casei transformed with pHCE2LB:pgsA-SARS SA. In FIG. 5B, lane 1 is non-transformed Lactobacillus casei, and lane 2, 3, 4, 5 and 6 are Lactobacillus casei transformed with pHCE2LB:pgsA-SARS SC/. In FIG. 5C, lane 1 is non-transformed Lactobacillus casei, and lane 2 is Lactobacillus casei transformed with pHCE2LB:pgsA-SARS SBC.

As shown in FIG. 5, specific fusion proteins [pgsA-SARS SA of about 54kDa (FIG. 5A), pgsA-SARS SC of about 51kDa (FIG. 5B) and pgsA-SARS SBC of about 78 kDa (FIG. 5C)] were identified in the whole cell of respective lactic acid bacteria.

Also, in order to confirm if respective antigen proteins were expressed with pgsA in the lactic acid bacteria transformed by the pHCE2LB:pgsA-SARS SA and pHCE2LB:pgsA-SARS:SBC surface expression vectors on the surface, the lactic acid bacteria transformed by the respective vectors were fractionated by the cell fractionation method using a ultracentrifuge into the cell wall and the cytoplasm and the positions of the respective fusion proteins were identified by Western blot using the specific antibody to pgsA.

Concretely, Lactobacillus which had the surface expression of the fusion proteins induced by the above described method were harvested to be the same cell concentration as non-transformed Lactobacillus. The cells were washed several times with TES buffer (10 nM Tris-HCl, pH 8.0, 1 mM EDTA, 25% sucrose), suspended in distilled water containing 5 mg/ml lysozyme, 1 mM PMSF and 1 mM EDTA, frozen at −60° C. and thawed at room temperature several times, treated with, DNase (0.5 mg/ml) and RNase (0.5 mg/ml) and subjected to sonication for cell destruction. Then, the cell lysate was centrifuged at 4° C., for 20 minutes at 10,000× g to separate the non-lysed whole Lactobacillus (pellet; whole cell fraction) and cellular debris (supernatant). The separated cellular debris was centrifuged at 4° C. for 1 hour at 21,000× g to obtain the supernatant (soluble fraction) containing cytoplasm proteins of Lactobacillus and pellets. The obtained pellets were suspended in TE solution (10 mM Tris-HCl, pH 8.0, 1 mM EDTA, pH 7.4) containing 1% SDS to obtain cell wall proteins (cell wall fraction) of Lactobacillus.

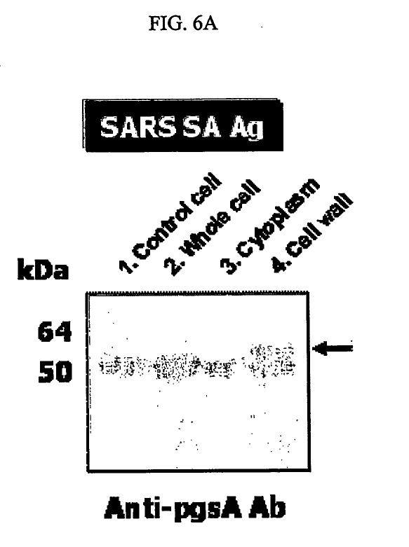

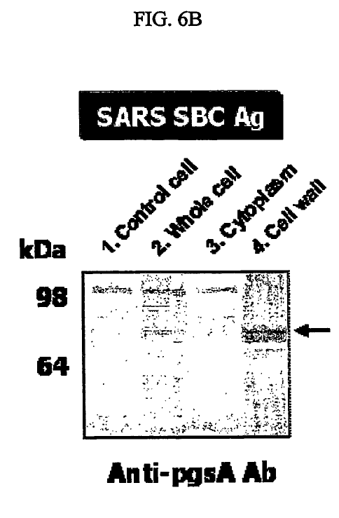

The respective fractions were subjected to Western immunoblotting using SDS-polyacrylamide gel electrophoresis and the antibody to pgsA antigen to confirm that the spike antigens of SARS virus fused with pgsA existed in the cell wall, among the respective Lactobacillus fractions (FIG. 6). In FIG. 6A, lane 1 is non-transformed Lactobacillus casei, lane 2 is the whole cells of Lactobacillus casei transformed with pHCE2LB:pgsA-SARS SA, lane 3 and 4 are the soluble fraction and the cell wall fraction of the strain trasformed with pHCE2LB:pgsA-SARS SA, respectively. In FIG. 6B, lane 1 is non-transformed Lactobacillus casei, lane 2 is the whole cells of Lactobacillus casei transformed with pHCE2LB:pgsA-SARS SBC, lane 3 and 4 are the soluble fraction and the cell wall fraction of the strain trasformed with. pHCE2LB:pgsA-SARS SBC, respectively.

As shown in FIG. 6, the SARS SA protein of about 54 kDa fused with pgsA and the SARS SBC protein of about 78 kDa fused with pgsA were identified in the whole cell and the cell wall fraction of lactic acid bacteria. From these results, it was noted that the respective SARS antigen proteins fused with pgsA were expressed and placed by migrating to the surface of lactic acid bacteria by pgsA.

Also, by fluorescence-activating cell sorting (FACS) flow cytometry, it was identified that the expression of the antigen group of the spike antigen of SARS virus took place on the surface of Lactobacillus by the fusion with C-terminal of the poly-gamma-glutamic acid synthesizing protein pgsA.

For immunofluorescence dying, expression induced Lactobacillus was harvested to be the same cell concentration. The cells were washed several times with buffer solution (PBS buffer, pH 7.4), suspended in 1 ml of buffer solution containing 1% bovine serum albumin and reacted with mouse-derived polyclone primary antibody to the spike antigen of SARS virus, which have been diluted 1000 times, at 4° C. for 12 hours. After completion of the reaction, the cells were washed several times with buffer solution, suspended in 1 ml of buffer solution containing 1% bovine serum albumin and reacted with biotin-binding secondary antibody, which have been diluted 1000 times, at 4° C. for 3 hours. Again, after completion of the reaction, the cells were washed several times with buffer solution, suspended in 0.1 ml of buffer solution containing 1% bovine serum albumin and bound to streptavidin-R-phycoerythrin dye agent specific to biotin, which have been diluted 1000 times.

After completion of the reaction, Lactobacillus was washed several times, and examined by fluorescence-activating cell sorting (FACS) flow cytometry. It was noted that as compared to non-transformed Lactobacillus, the SBC spike antigen protein of SARS virus was expressed on the surface of Lactobacillus (FIG. 6C). In FIG. 6C, the grey part is derived from non-transformed Lactobacillus casei and the white part is derived from transformed pHCE2LB:pgsA-SARS SBC/Lactobacillus casei. As shown in FIG. 6C, it was clearly noted that the SBC spike antigen protein was surface expressed in lactic acid bacteria transformed with pHCE2LB:pgsA-SARS SBC vector while no fluorescence expression was observed in non-transformed Lactobacillus casei.

### EXAMPLE 8

### Confirmation of Surface Expression of SARS Virus Nucleocapsid Antigen Protein on Lactic Acid Bacteria

Lactobacillus was transformed with the surface expression vectors pHCE2LB:pgsA-SARS NB and pHCE2LB:pgsA-SARS N and examined for expression of respective antigen proteins.

The expression of the antigenic sites in the nucleocapsid antigen of SARS virus fused respectively with the C-terminal of the gene pgsA synthesizing poly-gamma-glutamic acid was induced by transforming Lactobacillus casei with pHCE2LB:pgsA-SARS NB and pHCE2LB:pgsA-SARS N respectively, subjecting the transformed strain in MRS medium (Lactobacillus MRS, Becton Dickinson and Company Sparks, USA), to a stationary culture and multiplication at 37° C.

In order to confirm if respective antigen proteins were expressed with pgsA in the lactic acid bacteria transformed by the pHCE2LB:pgsA-SARS NB and pHCE2LB:pgsA-SARS N surface expression vectors on its surface, the lactic acid bacteria transformed with each vector by the same method as in the Example 7 were fractionated by the cell fractionation method using a ultracentrifuge into the cell wall and the cytoplasm and the positions of the respective fusion proteins were identified by Western blot using the specific antibody to pgsA.

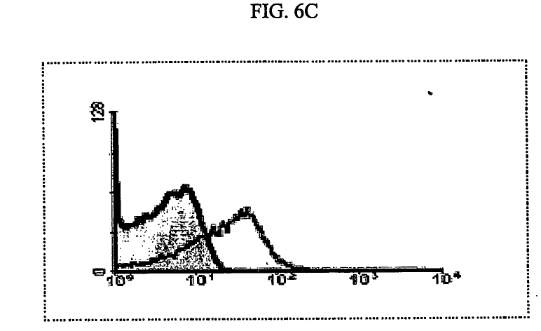

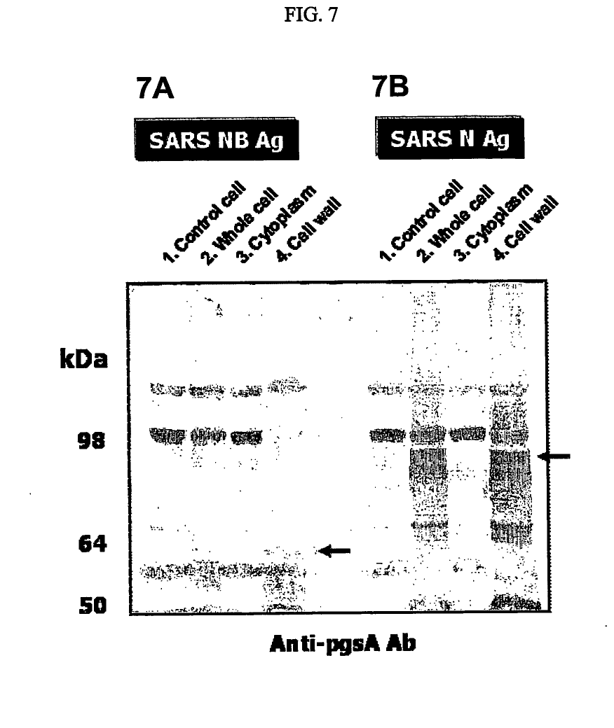

As a result, The respective fractions were subjected to Western immunoblotting using SDS-polyacrylamide gel electrophoresis and the antibody to pgsA antigen to confirm that the nucleo antigens of SARS virus fused with pgsA existed in the cell wall, among the respective Lactobacillus fractions (FIG. 7). In FIG. 7A, lane 1 is non-transformed Lactobacilius casei, lane 2 is the whole cell of transformed pHCE2LB:pgsA-SARS NB/Lactobacillus casei, lane 3 and 4 are the soluble fraction and the cell wall fraction of the strain trasformed with pHCE2LB:pgsA-SARS NB, respectively. In FIG. 7B, lane 1 is non-transformed Lactobacillus casei, lane 2 is the whole cell of the transformed pHCE2LB:pgsA-SARS N/Lactobacillus casei, lane 3 and 4 are the soluble fraction and the cell wall fraction of the strain trasformed with pHCE2LB:pgsA-SARS N, respectively.

As shown in FIG. 7, the SARS NB protein of about 57 kDa fused with pgsA and the SARS N protein of about 75 kDa fused with pgsA were identified in the whole cell and the cell wall fraction of lactic acid bacteria. From these results, it was noted that the respective SARS antigen proteins fused with pgsA were expressed and placed by migrating to the surface of lactic acid bacteria by pgsA.

### EXAMPLE 9

### Analysis of Vaccine Effect of Lactic Acid Bacteria with Spike Antigen Protein and Nucleocapsid Antigen Protein of SARS Virus Surface Expressed

Gram positive bacterium Lactobacillus casei was transformed with the surface expression vectors pHCE2LB:pgsA-SARS SA, pHCE2LB:pgsA-SARS SC and pHCE2LB:pgsA-SARS NB, prepared in the foregoing Examples and expression of the antigens on the surface of Lactobacillus casei was induced. The antigenicity of the spike antigen protein and nucleocapsid antigen protein of SARS virus fuged with cellular outer membrane protein pgsA participating poly-gamma-glutamic acid synthesis was examined using a mouse model.

Concretely, Lactobacillus casei was transformed with the surface expression vectors pHCE2LB:pgsA-SARS SA, pHCE2LB:pgsA-SARS SC and pHCE2LB:pgsA-SARS NB according to the present invention. The cells were harvested to be the same cell concentration and washed several times with buffer solution (PBS buffer, pH 7.4). 5×10⁹ Lactobacillus cells with the antigen surface expressed were orally administered to a 4-6 week old BALB/c mouse 3 times a day every other day, 3 times a day every other day after 1 week, 3 times a day every other day after 2 weeks, and 3 times a day every other day after 4 weeks. Also, 1×10⁹ Lactobacillus cells with the antigen surface expressed were intranasally administered to a mouse 3 times a day every other day, 3 times a day every other day after 1 week, 2 times a day every two days after 2 weeks, and 2 times a day every two days after 4 weeks. After oral and intranasal admistrations, every two weeks, \textcircled{1} serum of each mouse was taken and examined for IgG antibody value to the spike antigen protein and the nucleocapsid antigen protein in the serum and \textcircled{2} the suspension which comes after washing the inside of the intestines from each mouse and suspension which comes after washing the inside of bronchus and alveola from each mouse were examined for IgA antibody value to the spike antigen protein and nucleocapsid antigen protein, by ELISA.

10 BALB/c mice(4-6 week old) were assigned to one group. A mixture of lactic acid bacteria, each expressing SARS SA and SARS SC, was assigned to one group, lactic acid bacteria expressing SARS NB was assigned to one group, and a mixture of lactic acid bacteria, each expressing SARS SA, SARS SC and SARS NB, was assigned to one group. These three groups were divided into a oral administraion group and an intranasal administration group to make 8 groups including control group.

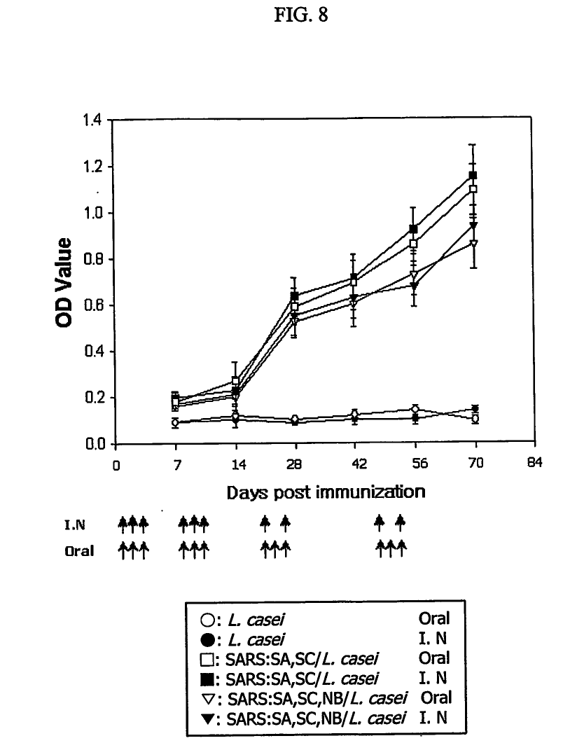

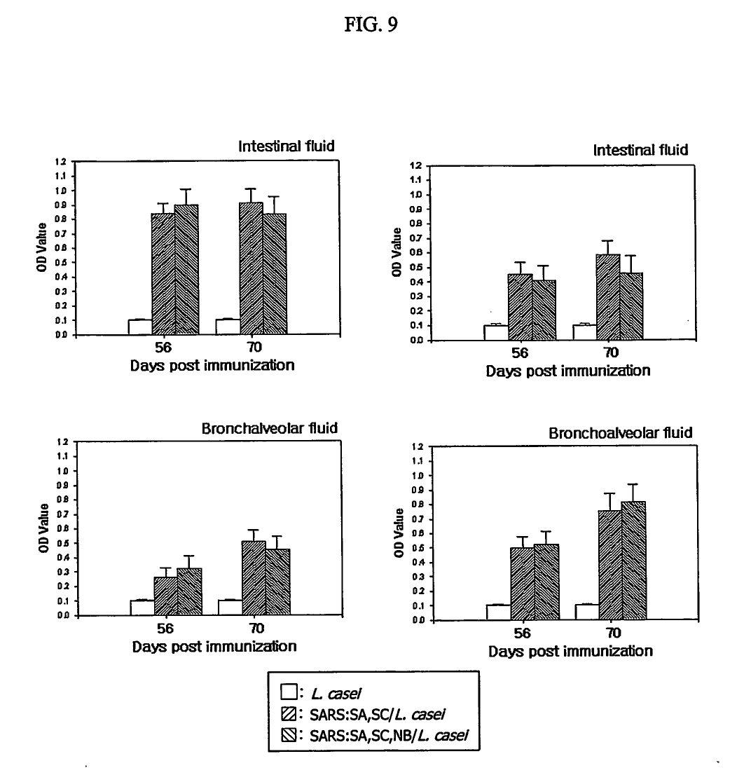

FIG. 8 shows the IgG antibody value to the SARS SA and SARS SC antigens, which are the spike antigen proteins of SARS virus, in serum of mice. FIG. 9 shows the IgA antibody value to the SARS SA and SARS SC antigens, which are the spike antigen proteins, in the suspension which comes after washing the inside of the intestines and suspension which comes after washing the inside of bronchus and alveola of mice according to ELISA, in which A is the IgA antibody value of the oral administration group and B is the IgA antibody value of the intranasal administration group.

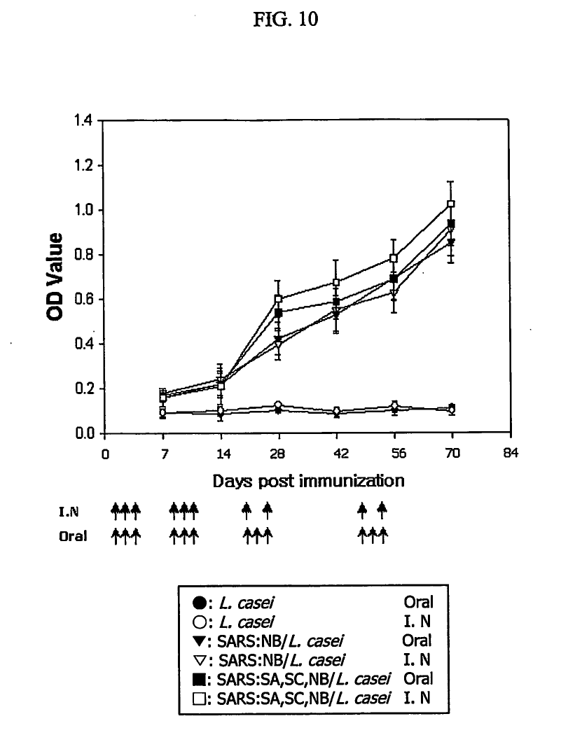

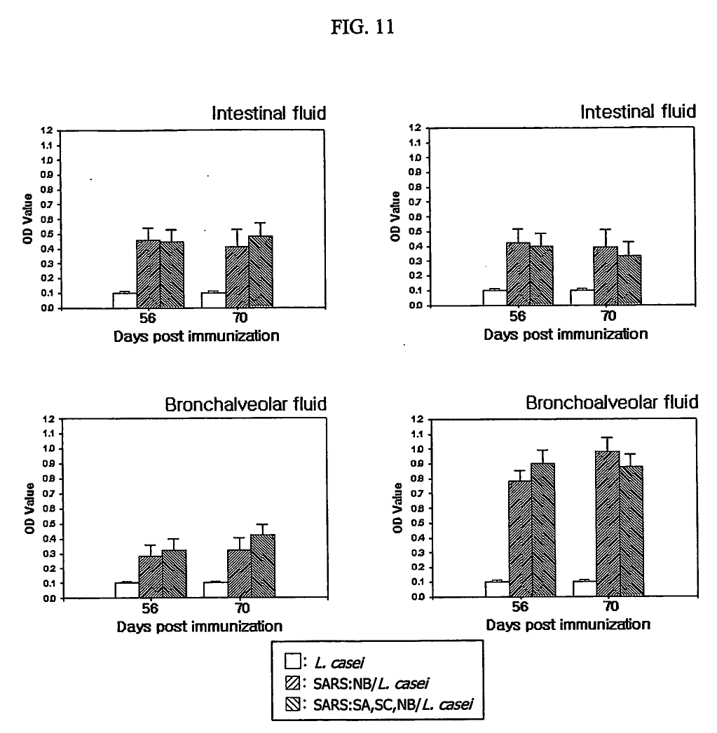

Also, FIG. 10 shows the IgG antibody value to the SARS NB antigen, which is the nucleocapsid antigen protein of SARS virus, in serum of mice. FIG. 11 shows the IgA antibody value to the SARS NB antigen, which is the nucleocapsid antigen protein of SARS virus, in the suspension which comes after washing the inside of the intestines and suspension which comes after washing the inside of bronchus and alveola of mice according to ELISA, in which A is the IgA antibody value of the oral administration group and B is the IgA antibody value of the intranasal administration group.

As shown in FIGS. 8 to 11, it was noted that the IgG antibody value and the IgA antibody value to the antigen groups of the spike and nucleocapsid antigen proteins of SARS virus were considerably higher in in the serum, the intestine washing liquid and bronchus-alveola washing liquid of BALB/c mice administered with transformed Lactobacillus by pHCE2LB:pgsA-SARS SA, pHCE2LB:pgsA-SARS SC and pHCE2LB:pgsA-SARS NB, alone or in combination as compared to the control group.

Therefore, it was noted that the microorganism having the antigen groups of the spike and nucleocapsid antigen proteins of SARS virus surface expressed according to the present invention can be effectively used as a live vaccine.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

### INDUSTRIAL APPLICABILITY

As described above, the transformed microorganism expressing an antigen protein of SARS inducing coronavirus on their surface according to the present invention and the antigen protein extracted and purified from the microorganism can be used as a vaccine for prevention and treatment of SARS. Particularly, it is advantageously possible to economically produce a vaccine for oral use using the recombinant strain expressing an SARS coronavirus antigen according to the present invention.

1. A surface expression vector comprising any one or two or more of pgsB, pgsC and pgsA genes encoding poly-gamma-glutamic acid synthase complex and a gene encoding a spike antigen protein or a nucleocapsid antigen protein of SARS coronavirus.

2. The surface expression vector according to claim 1, wherein the spike antigen protein is SARS SA, SARS SB, SARS SC, SARS SD or SARS SBC.

3. The surface expression vector according to claim 1, wherein the nucleocapsid antigen protein is SARS NA, SARS NB or SARS N.

4. The surface expression vector according to claim 2, wherein the vector is pHCE2LB:pgsA-SARS SA, pHCE2LB:pgsA-SARS SC or pHCE2LB:pgsA-SARS SBC.

5. The surface expression vector according to claim 3, wherein the vector is pHCE2LB:pgsA-SARS NB or pHCE2LB:pgsA-SARS N.

6. A microorganism transformed by the expression vector of claim 1.

7. The microorganism according to claim 6 wherein the microorganism is selected from the group consisting of E. coli, Salmonella typhi, Salmonella typhimurium, Vibrio cholerae, Mycobacterium bovis, Shigella, Bacillus, lactic acid bacterium, Staphylococcus, Listeria monocytogenes, and Streptococcus.

8. A method for producing a spike antigen protein or a nucleocapsid antigen protein of SARS coronavirus comprising culturing the microorganism of claim 6.

9. A vaccine for prevention of SARS virus comprising the spike antigen protein or the nucleocapsid antigen protein or the produced by the method of claim 8, as an effective ingredient.

10. The vaccine according to claim 9, wherein the antigen protein is an expressed form on the surface of microorganism, a crudely extracted form or a purified form.

11. The vaccine according to claim 9, wherein the vaccine is adapted to be taken oral administration or in food.

12. The vaccine according to claim 9, wherein the vaccine is adapted for subcutaneous or intra-peritoneal injection.

13. The vaccine according to claim 9, wherein the vaccine is adapted for intranasal administration.

14. The method according to claim 8, wherein the microorganism is lactic acid bacterium.

15. A lactic acid bacterium, which is produced by the method of claim 14 having the spike antigen protein or the nucleocapsid antigen protein of SARS coronavirus expressed on its surface.

16. A vaccine for prevention of SARS comprising the lactic acid bacterium of claim 15, an antigen protein extracted from said lactic acid bacterium, as an effective ingredient.

17. The vaccine according to claim 16, wherein the vaccine is adapted to be taken by oral administration or in food.

18. The vaccine according to claim 16, wherein the vaccine is adapted for subcutaneous or intra-peritoneal injection.

19. The vaccine according to claim 16, wherein the vaccine is adapted for intranasal administration.
